(19) United States
(12) Patent Application Publication
(10) Pub. No.: US 2008/0187568 A1
(43) Pub. Date: Aug. 7, 2008

(54) Polymerization with precipitation of proteins for elution in physiological solution

(76) Inventor: Amarpreet S. Sawhney, Lexington, MA (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 12/012,808

(22) Filed: Feb. 6, 2008

Related U.S. Application Data
(60) Provisional application No. 60/899,898, filed on Feb. 6, 2007.

(51) Int. Cl. A61K 47/30

(52) U.S. Cl. 424/422; 514/772.3

(57) ABSTRACT

Methods and compositions for polymerizing materials with the process of making the material causing precipitation of an agent that is subsequently released by the material. In some aspects, controlled delivery of bioactive substances from biodegradable hydrogels is disclosed. In certain embodiments, biodegradable hydrogels formed in situ comprise entrapped precipitated proteins that are ordinarily water soluble. The dissolving and release of the bioactive substance is impeded by the hydrogel structure and takes place at a slow rate, thus controlling the release of said bioactive substance from the hydrogel. Thus a bioactive substance that would normally be rapidly dissolved in a physiological surrounding is, instead, released in a controlled manner. The hydrogel phase also serves to protect the bioactive agent from cells and enzymes present in physiological surroundings.

### CROSS REFERENCE

This application claims priority to U.S. Ser. No. 60/899,898 filed Feb. 6, 2007, which is hereby incorporated by reference herein.

### TECHNICAL FIELD

The technical field relates generally to the field of polymer chemistry, and more particularly to the formation of hydrogels using water-soluble polymers and precursors with the polymerization process precipitating an agent within the hydrogel.

### BACKGROUND

Polymerization processes are known for forming hydrogels and crosslinked materials. The chemical and physical properties of precursors that form the hydrogel and the polymerization conditions affect the properties of the resultant material. Hydrogels can be made that subsequently release agents that interact with the hydrogel's environment.

For instance, for many diseases, a localized delivery of agents such as bioactive substances or drugs is highly beneficial. The localized delivery of a drug reduces systemic toxicity, but achieves high local concentration of the drug. Examples of such approaches include the local delivery of drugs to the cancerous tumors or treatment of restenosis using a drug coated stent. In many instances, it is highly desirable to treat many local diseases using minimally invasive surgical procedures with a biodegradable device or carrier. It is also useful to control the release of drug in a predictable fashion. Controlled release of bioactive agents or drugs is also preferred in some cases over a bolus release due to the maintenance of a minimum therapeutic concentration over a longer period and thus avoiding repeated dosing while providing for more efficient utilization of the drug.

Several types of non degradable and degradable implant systems have been described in the literature for local and controlled drug delivery. Degradable polymers such as poly(lactic acid), poly(glycolic acid) and their copolymers, along with several other degradable polymers have been described for local and controlled drug delivery. However, these polymers create acidic species upon breakdown in the body and may denature sensitive drugs such as growth factors, while also creating local inflammation at the site of implantation. They are also not easily administered through less invasive means, since they are either only soluble in organic solvents, which are not bioinert, or are present as a solid form. Biodegradable hydrogels are also known that can be used to administer various drugs.

### DETAILED DESCRIPTION OF PREFERRED EMBODIMENTS OF THE INVENTION

A polymerization system can be controlled to precipitate agents during the polymerization of a material. As the material's environment changes, the conditions in the material change and the agent can be released so that the material is responsive to its environment. Bioactive substances can be precipitated in situ in a patient at the same time as hydrogels are formed that entrap them. The hydrogels create a chemical environment that forms the precipitates. The precipitated substances can redissolve as the hydrogel interacts with its physiological surroundings by reaching a new chemical balance with physiological fluids. The new chemical balance can change over time as the hydrogel responds to its environment by dissolving, being degraded, or chemically interacting. All of these forces serve to control delivery of the substance to the body.

In some aspects, the bioactive substance may thus be dissolved, or dissolvable, at a first concentration and is in a condition of readiness to be taken up into the body but its solubility is limited by the introduction of the hydrogel components into its chemical environment. Then the substance has only a limited solubility and can be present in solution only at a second concentration that is less than the first concentration, so its release is controlled. Controlling release of such agents is a challenging problem, but these techniques allow for the solubility of the agent to be engaged to control the release to achieve an important improvement over conventional technology. Accordingly, a bioactive substance may be dissolved at a first concentration, exposed to components of a hydrogel system that cause the substance to precipitate, and then controllably released into its environment by redissolving.

One advantage of this approach is that the hydrogel precursors and polymerization process can be chosen so that polymerization changes the environment of an agent to cause precipitation of a wide selection of agents such as bioactive substances. Further, a single hydrogel system may be used to deliver many drugs, so that multiple drugs may readily be delivered simultaneously. Moreover, this standardization provides for a standard clinically-accepted hydrogel to be used as a platform for many substances. A material that is standard for a broad set of agents is an important improvement over alternatives that require custom-tailoring of a hydrogels for each agent. Another advantage is that the precipitation mechanism is quite simple because the system can be configured to revolve around the solubility of the substance. In contrast, an abundance of other schemes use complicated material structures or chemical combinations with the substance to attempt to achieve controlled release.

### Precipitation

Precipitation is the formation of a solid in a solution during a chemical reaction. The solid formed at the time of the reaction is called the precipitate. The formation of a precipitate is a sign of a chemical change. As customarily used in chemical processes, the solid forms and falls out of the solute phase, and can be collected from the solution by various methods, e.g., filtration, decanting, or centrifuging.

Various factors can affect solubility and precipitation. For instance, volume exclusion effects, salt concentration, solvent properties, ion content, and pH can each have an effect. All of these effects can be mixed and matched as needed to produce a suitable technique for the substance that is to be delivered in light of the hydrogel or other delivery system that is to be used.

One precipitation technique is an excluded volume process based on using solutes to concentrate a substance. A substance dissolved in a solvent is concentrated by adding other solutes that occupy a volume of the solvent to the exclusion of the substance, so that the substance is concentrated in the solvent. When the substance's concentration exceeds its solubility, it typically precipitates. It is known in the scientific literature of protein separation science that proteins can be separated by the use of water soluble polymers such as polyethylene glycol (PEG), dextrans, polyvinyl pyrrolidinone, polyvinyl pyrrolidinone, various hydrophilic polymers, and the like.

An example of the excluded volume process is the precipitation of proteins from aqueous solution by using PEG, see Atha and Ingham, J. Biol. Chem., 256(23): 12108-12117 (1981), which is hereby incorporated herein by reference. PEG or PEO is a term used for polymer containing ethylene oxide repeat units. In general, protein solubility in the presence of PEG follows the equation log S=bC+x, with S being solubility and C being PEG concentration. The chemical interactions between attractive and repulsive forces between PEG and proteins are, in general, relatively unimportant in the precipitation mechanism. And temperature typically does not have a significant effect. PEGs are somewhat more effective in causing precipitation as their molecular weight (MW) increases. And proteins are somewhat more sensitive to PEG as they increase in MW. Thus PEG can be used to precipitate a wide variety of proteins and other substances.

The separations using PEG and other polymers are dependent on concentration of the polymer, its molecular weight and type of polymer use. The polymers for the precipitation can be chosen as needed considering the guidance herein. Polyethers and polyalkylene oxides are useful for precipitations. Polyethers include PEG and polypropylene glycol (PPG). Depending on the polyether, polyalkylene oxides, or PEG derivatives used, a preferred molecular weight range is about 200 to about 100,000, or more preferably about 400 to about 5,000, e.g., about 2,000; artisans will immediately appreciate that all the ranges and values within the explicitly stated ranges are contemplated. In general, a higher concentration of precipitating polymer generally produces more effective separation. Typically, for preferred polyether, polyalkylene oxides, or PEG derivatives, concentration is about 10 to about 70% w/w, or more preferably about 20 to about 60% w/w range. Further, polyether, polyalkylene oxides, or PEG derivatives can be used to make copolymers for precipitations, e.g., a PLURONICS polymer. In some embodiments, the copolymer is at least about 40% by molecular weight polyether, polyalkylene oxide, or PEG, e.g., 100%, at least about 50%, or 60%; artisans will immediately appreciate that all the ranges and values within the explicitly stated ranges are contemplated.

Another precipitation technique is referred to as salting-out. Protein solubility is a function of the physiochemical nature of the proteins, pH, temperature and the concentration of the salt used. It also depends on whether the salt is kosmotropic (stabilizes water structure) or chaotropic (disrupts water structure). At low concentrations of salt, solubility of the proteins usually increases slightly (salting in). Initial salting in at low concentrations is explained by the Debye-Huckel theory. Proteins are surrounded by the salt counter ions (ions of opposite net charge) and this screening results in decreasing electrostatic free energy of the protein and increasing activity of the solvent, which in turn, leads to increasing solubility. This theory predicts the logarithm of solubility to be proportional to the square root of the ionic strength.

But at high concentrations of salt, the solubility of the proteins drop sharply (salting out). The abundance of the salt ions decreases the solvating power of the salt ions so that the solubility of the proteins decreases and precipitation results. At high salt concentrations, the solubility is given by the empirical expression: log S=B−KI where S is the solubility of the protein, B is a constant (function of protein, pH, and temperature), K is the salting out constant (function of pH, mixing and salt), and I is the ionic strength of the salt. As different proteins have different compositions of amino acids, different protein molecules precipitate at different concentrations of salt solution.

Certain ions can increase the solubility of a protein when the concentration of the ions increases, instead of decreasing the solubility of the protein. Also some ions can denature certain proteins so if assays on the function of proteins are intended then either a different ion or an alternative purification method should be used. Ammonium sulfate precipitation is a method of protein purification by altering solubility of protein. Ammonium sulfate is commonly used as its solubility is so high that salt solutions with high ionic strength are allowed.

Thus biocompatible salts or low molecular weight compounds such as sucrose, fructose, glucose, sodium chloride, glycerol, potassium chloride, sodium phosphate and potassium phosphate, triethanol amine, triethanol amine hydrochloride, magnesium chloride and their combinations and the like may also be used to precipitate a protein or other bioactive substance. Biocompatible is a term used to describe acceptable tissue interaction with the organism or the implant site of the polymer. Typically, salts such as sodium chloride may be used in the concentration range from 5% to 30%, more preferably 15% to 30% range. Combination of two or more salts such as sodium chloride (30%) and sucrose (10 to 45%) may be used to keep the protein in the solid state or prevent its dissolution. A salt or compound that will not interfere in the gelation process is most preferred. For example, sucrose or sodium chloride which does not have functional groups capable of reacting with polyamine based precursors or macromonomers are most preferred. Some additives such as sucrose at high concentration (>30%) produce high viscosity solutions. High viscosity solutions may not be desirable when delivered using catheter techniques or for certain spraying applications. Under such conditions, salt like compounds such as sodium chloride may be used. The use of sodium chloride does not affect the viscosity of the medium in a significant manner. In one embodiment, a 20% solution of sodium chloride and 25% sucrose is used to dissolve dilysine or a macromonomer at low concentration.

In some embodiments, solvent properties are used to control precipitation. Some materials are nonsoluble or only sparingly soluble in aqueous solution but can be dissolved in organic solvents, or with a mixture of aqueous and organic solvents. Some organic solvents are relatively more biocompatible than other solvents, e.g., dimethyleformamide or some low MW alcohols. The bioactive substance can be dissolved in the organic solvent or organic-aqueous solvent mixture, and then exposed to another solution so that the concentration of organic solvent is decreased below the point of solubility and the substance is precipitated. Examples of alcohols are methanol, ethanol, propanols, butanols, and molecules that comprise a C₃ or longer alkyl and an —OH, with the —OH optionally being on the alkyl or optionally terminating the alkyl; accordingly, alkyls such as a C₄ or C₆ may be used.

In some embodiments, ion content or pH is used to control precipitation.

### In situ Hydrogel Polymerization

Hydrogels can be formed in situ from one or more precursor molecules. A hydrogel refers to a solid crosslinked insoluble hydrophilic material holding a substantial amount of water. Crosslinking refers to the method of forming covalent bonds or crosslinks either between polymeric/macromolecular molecules. A crosslinking agent is as a compound capable of forming crosslinks. In situ refers to a local site in a patient's body wherein the hydrogel is intended to be placed and to remain for its functional life, and is typically in contact with a tissue. In some embodiments, two or more precursors are mixed to initiate crosslinking to form the hydrogel; the hydrogel is thus comprises of the precursors, which are components of the hydrogel. Polymerization is a broad term that includes reacting precursors by electrophilic-nucleophilic reactions to form a crosslinked material having an abundance of repeats of the precursors. In other embodiments, one or more precursors are activated to begin a process that includes crosslinking, e.g., by initiation of a free radical polymerization. In situ formed biodegradable hydrogels may be administered in a liquid or fluent state and then transformed into a solid at the site of application. This transformation may be done using external energy, such as photopolymerization, or spontaneously, using a chemically initiated polymerization. The process may also be performed in the absence of external energy application (e.g., no light activation), or in the absence of free radical initiators, or be triggered merely by mixing components of the system (e.g., electrophilic-nucleophilic reactions).

In situ formation may take place by any suitable means, with minimally invasive surgery techniques being useful in many modern surgeries. Some embodiments call for precipitation of an agent simultaneously with another event such as mixing or polymerization; in this context, the term simultaneous means close in time and causally related. For instance, a mixing process may take an amount of time to change a solvent's properties to thereby cause precipitation, but is nonetheless effectively simultaneous. Thus, for some embodiments directed to simultaneous mixing and polymerization, the polymerization may start with mixing and be completed later. Some embodiments are directed to a combination of mixing and deposition at a location; the mixing may be accomplished prior to, simultaneous with, or at about the time of deposition. Accordingly, a sprayer that mixes and'deposits precursors to form a material at a location may cause polymerization to start before, during, or at about the time of deposition, bearing in mind that some applications call for components that begin a polymerization when mixed.

The term minimally invasive surgery or (MIS) is used herein includes surgical techniques such as laparoscopy, thoracoscopy, arthroscopy, intraluminal endoscopy, endovascular techniques; catheter based cardiac techniques (such as balloon angioplasty) and interventional radiology. Minimally invasive techniques also contemplate injection through fine needles or through needleless injector systems.

The hydrogels may be biodegradable into small pieces, e.g., by spontaneous hydrolysis in water, i.e., without enzymatic intervention, for instance, the cleavage of ester groups by exposure to water. In general, the biodegradation results in the material breaking down into smaller components that are then cleared by the body, for instance, small molecules that are cleared by the kidneys. Other materials may be degraded by enzymatic or cellular-based degradation, e.g., most proteins. Biodegradable hydrogels are materials that can demonstrate an increased biocompatibility over other biodegradable polymers, since the byproducts of the hydrogel degradation can tend to be non-inflammatory.

Certain biodegradable hydrogels that can be polymerized in situ are described in U.S. Pat. No. 5,410,016, which describes compositions and methods for formation of biodegradable hydrogels by free radical polymerizations. Polymerize means molecules are reacted that have the capacity to form additional covalent bonds resulting in monomer interlinking, for example, molecules containing carbon-carbon double bonds of acrylate-type molecules or molecules that have functional groups that can interact with other molecules having complementary functional groups that can form a covalent bond when exposed to each other under suitable circumstances. Biodegradable is a term used for those crosslinkers, gels, polymers and macromolecules which degrade or hydrolyze inside the human or animal body.

Another reference is U.S. Pat. No. 6,566,406, which describes, among other things, formation of biodegradable hydrogels by condensation polymerization or a reaction between electrophilic or nucleophilic precursors is used to form biodegradable hydrogels. Both patents teach methods of their formation and controlling their degradation times. Other methods of biodegradable hydrogels include gels that are formed as a result of temperature changes, e.g., gels such as Pluronic gels or hydrogels that are formed when delivered using water miscible organic solvents. Other references that describe in situ hydrogel formation are U.S. Pat. Nos. 5,874,500, 6,152,943, 6,514,534, 6,605,294, 6,632,457, 6,818,018, and 6,887,974.

Polymerization may take place by electrophilic functional groups reacting with nucleophilic functional groups to form covalent bonds. The functional groups may be, e.g., electrophiles reactable with nucleophiles, groups reactable with specific nucleophiles, e.g., primary amines, groups that form amide bonds with a protein, groups that form amide bonds with carboxyls, activated-acid functional groups, or a combination of the same. An electrophile may be of a type that does not participate in a Michaels-type reaction or of a type that participates in a Michaels-type reaction. A Michael-type reaction refers to the 1,4 addition reaction of a nucleophile on a conjugate unsaturated system. The term conjugation can refer both to alternation of carbon-carbon, carbon-heteroatom or heteroatom-heteroatom multiple bonds with single bonds, or to the linking of a functional group to a macromolecule, such as a synthetic polymer or a protein. Michael-type reactions are discussed in detail in U.S. Pat. No. 6,958,212, which is hereby incorporated by reference for all purposes to the extent it does not contradict what is explicitly disclosed herein. Examples of electrophiles that do not participate in a Michaels-type reaction are: succinimides, succinimidyl esters, NHS-esters, or maleimides. Examples of Michael-type electrophiles are acrylates, methacrylates, methylmethacrylates, and other unsaturated polymerizable groups.

Examples of reactive functional groups on a precursor are n-hydroxysuccinimide (NHS), n-hydroxysulfosuccinimide, and maleimides. An advantage of the NHS-amine reaction, in particular, is that the reaction kinetics can be used to make mixtures that lead to quick gelation, usually within 10 minutes, within 1 minute or within 10 seconds. Gelation refers to a viscous state that can be achieved before polymerization is complete. The NHS-amine crosslinking reaction leads to formation of N-hydroxysuccinimide as a side product. The sulfonated or ethoxylated forms of N-hydroxysuccinimide have increased solubility in water and hence a rapid clearance from the body. The sulfonic acid salt on the succinimide ring does not alter the reactivity of NHS group with the primary amines. In some embodiments, an NHS-amine crosslinking reaction is carried out in aqueous solutions and in the presence of buffers. Examples are phosphate buffer (pH 5.0-7.5), triethanolamine buffer (pH 7.5-9.0) and borate buffer (pH 9.0-12) and sodium bicarbonate buffer (pH 9.0-10.0).

One embodiment of making a hydrogel involves using at least one multifunctional crosslinker. The term multifunctional refers to precursors with at least two reactive functional groups for forming covalent bonds. Crosslinkers may include, for example, 2, 3, 4, 5, 6, 7, 8, 9, 10, 11, or 12 functional groups, or more.

The in situ material may be made adhesive to a tissue by providing the precursor or precursors with a viscosity and fluidity that allows for penetration into the crevices and minute irregularites of the tissue surface while at the same time polymerizing the precursors to solidify them and create a tenacious structure that surrounds these irregularities. A flowable material advantageously conforms to the tissue to intimately coat it; in contrast, pre-fabricated devices can be placed onto a tissue to contact it, but lack the intimate conformity created by the in situ formation. Accordingly, an in situ formed material disposed on a tissue has a distinct structure compared to a not-in situ formed material. This distinct structure is a conformal surface, i.e., the surfaces whereupon the in situ formed material is made. The term tissue is broad and includes extracorporeal and extracorporeal body parts. Some embodiments are intended for moist tissues, e.g., a tissue inside a body (not touching the epidermis), an eye tissue (e.g., intraocular or corneal) or extracorporeal (not penetrating beyond the epidermis of the skin).

In situ formed materials can be used for a variety of purposes. One embodiment is the formation of a drug delivery depot, e.g., as an implant inside a body. Other uses are, e.g., surgical adhesives, glues, dressings, hemostatic agents, wound healing agents, or sealants. For example, precursors can be reacted with natural or synthetic polymers with reactive functional groups (with or without biodegradable groups) to form a crosslinked material. Monomers may also be used in a polymerization reaction to form crosslinked materials. Solvents may be combined with the crosslinkers, monomers, or macromers. Compositions that have no solvents, or are free of water, may also be formulated to make materials in situ. Where convenient, a crosslinked gel material may include a visualization agent (e.g., where a sealant is used in a laproscopic method). A variety of clinical applications may be used, e.g., as in Schlag & Redl, Fibrin Sealant in Operative Surgery (1986) Vol. 1-7, for example, cardiovascular surgery, orthopaedic surgery, neurosurgery, ophthalmic surgery, general surgery and traumatology, plastic reconstruction and maxillofacial surgery, otorhinolaryngology, and the like.

Some embodiments are directed to in situ formation of a material, which refers to forming a material at its intended site of use. Thus a hydrogel may be formed in situ in a patient at the site wherein the hydrogel is intended to be used, e.g., as a sealant, wound dressing, or drug depot for controlled release. If the material is a gel used as a surgical sealant, the crosslinked gel can be utilized in humans or in other animals. Medical applications for a sealant include, e.g., connecting tissue or organs, stopping bleeding, healing wounds, sealing a surgical wound. Or the crosslinked materials may be used for tissue engineering applications such as providing matrix for cell growth or coating of vascular grafts. The dosage of the composition will depend upon its intended use. In most surgical application applications 1 to 500 ml total volume of biological fluid (or other precursor fluid) and crosslinker introduced in situ will be sufficient but other volumes may be used as needed; artisans will immediately appreciate that all the ranges and values within the explicitly stated ranges are contemplated.

### Bioactive Substances for Precipitation

Bioactive substances are described in the patents referenced herein, and include bioactive substances (bioactive agents, therapeutic agents, drugs) that are water soluble (soluble in aqueous solution at a concentration of least 1 gram per liter), sparingly water soluble (soluble in aqueous solution at a concentration of least 0.01 gram per liter and less than 1 gram per liter), essentially not soluble in aqueous solution, hydrophobic, or those agents that have been modified with functional groups that enhance their water solubility so that they move from one of these categories to another.

In some embodiments, the bioactive substances are water soluble and are entrapped within an in situ polymerized hydrogel, so as to be present in a solid phase whose dissolution is impeded by the hydrogel structure that slowly dissolves and releases over an extended period of days to months under physiological conditions. Entrap refers to substantially surrounding the entrapped material; in the case of a precipitate in a hydrogel, the precipitate may be larger or smaller than the mean space between hydrogel strands. The preparation of the agent may be controlled to make particles of a desired size to accomplish the same. Processes that entrap a material generally entrap most of the material but some of the material may be left on or outside the hydrogel unless otherwise indicated. Various materials and methods are described herein for controlled drug delivery of bioactive substances wherein such compositions are generated in situ upon reconstitution of the bioactive agent or drug with the liquid precursors of the hydrogel or may be present as suspended or dispersed solids in their precursors, and may optionally be delivered using minimally invasive surgical procedure.

The terms medicinal agent is directed to those agents used for medical purposes, including diagnosis, medical visualization, or bioactive effect, and includes agents referred to herein as bioactive agents or bioactive substances. The term bioactive agent or bioactive substance is used to refer to those agents used for medical purposes to have a bioactive effect. Some of the examples of bioactive substances that can be released inside the human or animal body include antiviral agents; antiinfectives such as antibiotics; antipruritics; antipsychotics; cholesterol or lipid reducing agents, cell cycle inhibitors, anticancer agents, antiparkinsonism drugs, HMG-CoA inhibitors, antirestenosis agents, antiinflammatory agents; antiasthmatic agents; antihelmintics; immunosuppressives; muscle relaxants; antidiuretic agents; vasodilators such as nitric oxide or nitric oxide adducts; beta-blockers; hormones; antidepressants; decongestants; calcium channel blockers; growth factors such as bone growth factors, bone marrow proteins, vascular endothelial growth factor, platelet derived growth factor, acidic growth factors, basic growth factors, wound healing agents, analgesics and analgesic combinations; local anesthetics agents, antihistamines; sedatives; angiogenesis promoting agents; angiogenesis inhibiting agents; tranquilizers and the like. In many instances, the duration of the drug release is affected by the hydrophobicity of the drug. The amount of bioactive substance incorporated in the tissue composite materials is in the range of about 0.1 percent to about 90 percent, more preferably in the about 5 to about 70 percent range and even more preferably in the about 10 to about 50 percent range. In some cases, two or more bioactive substances may be used to achieve a desirable therapeutic effect.

### In situ Hydrogel Formation and Bioactive Substance Precipitation

The hydrogels may be formed in situ with the bioactive substances present such that the bioactive substances are precipitated at about the time of the hydrogel's formation. In general, the bioactive substance is placed into solution and the solution is mixed with at least one precursor of the hydrogel such that the substance is precipitated, either by the presence of the precursor itself, or by other changes in the substance's chemical environment, such as a volume exclusion effect, or a change in salt concentration, solvent properties, ion content, or pH.

In some embodiments, the precursor solution may include PEG and/or other factors that can cause precipitation of a bioactive substance. The precursor solution is combined with the bioactive substance, which is thereby precipitated. The precursor solution is also reacted to form the hydrogel, which entraps the precipitated bioactive substance.

Biodegradable hydrogels useful for controlled drug delivery and that can be formed in situ during a surgical procedure have been described previously, as in U.S. Pat. No. 5,410,016 that describes the use of photopolymerizable hydrogels for controlled drug delivery. This patent describes the use of crosslinking density to control the release of bioactive substances. And U.S. Pat. No. 6,566,406 describes the formation of biodegradable hydrogels using condensation polymerization reactions. These patents however, do not teach the precipitation of bioactive substances. There is a need for compositions and methods, especially biodegradable compositions and methods, which can deliver drugs that would ordinarily be water soluble in a physiological environment in a controlled manner and via techniques that are compatible with minimally invasive techniques. These are also a need for protecting such drugs from degradation by the physiological environment over this extended period of delivery.

### Bioactive Substance Delivery

In some embodiments, the biodegradable hydrogels are formed in situ and the hydrogels have one or more bioactive substances. The bioactive substance may be present in a separate phase, such as solid state. Hydrogels by definition contain large amounts of water and are not dissolved in water. The bioactive substances, for example, may be present as an amorphous or crystalline or semicrystalline solid or may be present as an insoluble liquid such as an oil or semisolid wax. The phase separation from biodegradable hydrogel may occur after formation of hydrogel in situ or may exist prior to in situ formation of the gels.

In some embodiments, the bioactive substance is precipitated using a precursor to the hydrogel (see Example 1). The bioactive substance is present as a solid and the hydrogel forms around it, with the substance being released over time. Alternatively, the substance is precipitated by another factor in a solution that is combined with other solutions in the process of making the hydrogel in situ.

Example 1 shows how PEG may be used to precipitate a bioactive substance and that substance may be a solid when the hydrogel is formed. The in situ polymerizable hydrogel is obtained by mixing two precursors containing electrophilic and nucleophilic groups. The electrophilic and nucleophilic groups react in situ to form a biodegradable hydrogel. A commercial example of such in situ polymerization system is DURASEAL or SPRAYGEL surgical sealant system commercialized by Confluent Surgical Inc. (Waltham, Mass.).

In general, the materials may be made at a location exposed to a physiological fluid. A physiological fluid refers to a fluid in a patient, e.g., a human or an animal. Such fluids include tears, saliva, and interstitial fluid. The location may be chosen to target delivery to one of these types of fluids, or other fluids, or fluids within a certain pH range, e.g., from pH about 7 to about 8. Other locations with a pH of less than 6 may be targeted, e.g., in the stomach. Accordingly, some embodiments are directed to locations outside the stomach or excluding stomach pH conditions. Other locations are alkaline with a pH for more than about 8 and may be targeted accordingly. A location exposed to a physiological fluid refers to a site in a patient that contacts a bodily fluid. In some cases, the location is chosen to avoid contact with a blood fluid. Thus extravascular deposition is an embodiment of the invention.

### General Principles as Exemplified by Specific Examples

Various examples are set forth to exemplify the general principles described herein. In general, the features of each embodiment may be mixed-and-matched with each other to form other embodiments that are operable.

In the exemplary embodiment described in Example 1, AVASTIN is used as model bioactive substance. Precursor A is prepared by dissolving PEG-NHS ester in PBS and precursor B is prepared by dissolving PEG-amine solution in PBS. AVASTIN or BEVACIZUMAB (Genentech Inc), a recombinant humanized monoclonal IgG1 antibody that binds to and inhibits the biologic activity of human vascular endothelial growth factor (VEGF). BEVACIZUMAB contains human framework regions and the complementarily-determining regions of a murine antibody that binds to VEGF. Bevacizumab has a molecular weight of approximately 149 kilodaltons and is considered as new class of drug commonly referred as antiangiogenesis drugs. This class of drugs are believed to be useful in cancer treatment and also for the treatment of ophthalmic disease like neovascular age-related macular degeneration as well as for macular edema. AVASTIN is added to solution B and it forms a suspension in the PEG solution. The solubility of protein drug is limited in PEG containing solution because PEG and protein compete with water molecules for hydrogen bonding and increase solubility. The presence of PEG prevents the protein drug AVASTIN from solubilizing in aqueous medium. AVASTIN stays in the precursor as a solid particulate (phase separation). The precursors are mixed in such a way that the stoichiometry between electrophilic and nucleophilic group is maintained. Molar equivalency of amine and NHS groups is helpful to promote rapid and complete polymerization and crosslinking. Upon mixing A and B, the PEG amine and PEG-NHS ester react in presence of AVASTIN particles and form a crosslinked hydrogel network. A crosslinked hydrogel matrix prepared by mixing A and B and containing AVASTIN is used to monitor the release of AVASTIN form the crosslinked hydrogels. The hydrogel is incubated in PBS solution at 37° C. over a period of several weeks and AVASTIN concentration in eluted samples is monitored by protein assay. In order to release AVASTIN from the hydrogel, the AVASTIN must solubilize in water and then diffuse out from the hydrogel matrix to the surrounding tissue for therapeutic action. The overall kinetics or release is controlled by the dissolution of AVASTIN in water as well as diffusion from the crosslinked hydrogel. The dissolution of AVASTIN is key component in controlling the release from the hydrogel and is also a new element.

This phase separation of AVASTIN is created by design. The phase separation has two important advantages. The phase separation permits slow dissolution of drug after in situ polymerization which helps to control the release and phase separation also reduces interference by the drug or bioactive substance in polymerization/crosslinking reaction. In example 1, phase separation is created by the use of polyethylene glycol or its derivatives. A salt/sugar presence in the solution may prevent dissolution of AVASTIN or fibrinogen in the polymerization system. The presence of PEG or salts keeps the AVASTIN in solid state during crosslinking and polymerization reaction. The chemistry of polymerization (pH, buffers, concentration and temperature), various electrophilic and nucleophilic groups, the control of degradation of crosslinked hydrogels are explained in the patents cited herein. The A and B solution described above can be deployed using MIS techniques to form a drug delivery device inside the human or animal body. The components of A and B can be provided as lyophilized powders and reconstituted just prior to using. Use of spray or other MIS technique may be used to transport the composition to a surgical site. The dose of bioactive substance can be controlled by the type or nature of drug used and disease condition being addressed. The bioactive substance may be mixed with solution A or B. The choice is determined by the reactivity of bioactive substance towards precursors used. Preferably, bioactive substance is mixed with a precursor which will not react with the bioactive substance under normal storage and use. For example, AVASTIN is least likely to react with amine groups of PEG amine precursor under normal storage and use conditions and therefore mixing with PEG amine is preferred. Those skilled in the art of polymer chemistry, pharmaceutical chemistry will be able to choose appropriate mixing conditions depending on the chemistry of precursors and bioactive substances. Similar to AVASTIN, other protein drugs could also be used. The polymerization conditions and buffer conditions may be used to precipitate or phase-separate the protein drugs. For example, certain basic and acidic growth factors are soluble in acidic or basic solutions respectively. Precursor reaction conditions such as buffers may be chosen and their concentrations may be adjusted to precipitate the protein drug. In one embodiment, bone growth factors which are soluble in highly acidic solutions, but insoluble in neutral or basic condition are used to suspend the drug. Bone growth factor is suspended in polyamine solution at high PH where growth factor forms a suspension. The suspension is the reacted with PEG-NHS ester to complete the polymerization and entrap the particles of bone growth factor. In separating or precipitating the drug, care is taken that the bioactive substance does not loose its desirable bioactivity due to denaturation of protein. Those skilled in the protein chemistry art will know that aggressive solvents, excessive pH, high temperatures can promote loss of biological activity. The preferred polymerization conditions are chosen experimentally where the biological activity of bioactive substances is maintained.

Commercially available paclitaxel formulations are often present in a water soluble or water dispersible form. If administered in such a form, it rapidly disperses in an aqueous environment. However, the low water solubility of Paclitaxel can be exploited to create a separate phase during in situ formation of implant. Commercially available paclitaxel formulations for intravenous administration can be mixed with trilysine derivative precursor (Example 7). The mixing of amine solution suspends the paclitaxel crystals in the solution. Upon mixing with PEG NHS ester groups, the polymerization and crosslinking takes place and the paclitaxel crystals are entrapped in the hydrogel. The paclitaxel crystals slowly dissolve and the solubilized paclitaxel then diffuses from the hydrogel network. In this case, paclitaxel being small molecular therapeutic, the elution kinetics is dominated by dissolution of the paclitaxel crystals and not by the diffusion of drug from hydrogel matrix. Other water insoluble drugs that could be used in place of paclitaxel include but not limited to are: statin drugs like ATORVASTATIN, SIMVASTATIN, CERIVASTATIN, antibiotics like chlorhexidene derivatives and the like.

In another embodiment, a water insoluble bioactive substance such as vitamin E is used as a model bioactive substance. Vitamin E (alpha-Tocopherol) or its derivatives exist as an oily liquid at ambient or physiological conditions. The mixing of vitamin E with amine precursor solution creates a water-oil suspension/emulsion which is then reacted with PEG-NHS ester to form a crosslinked hydrogel. The oily water insoluble phase remains entrapped in the hydrogel and is then released as it dissolves in the water.

In another exemplary embodiment, a thermosensitive hydrogel is used to release the AVASTIN. First a solution of Pluronic F-127 is made by dissolving 4 grams of Pluronic F127 solution in 6 grams of PBS. The mixture is cooled to less than 10 degree C. to dissolve the polymer. 100 mg of AVASTIN lyophilized powder is added to the cold solution. The drug remains as suspended particles due to presence of Pluronic polymer (a PEG based derivative). The cold solution is applied on the tissue where body temperature transforms the Pluronic solution into soft hydrogel. The drug is released from the hydrogel due to dissolution of drug crystals. Other thermosensitive sensitive systems based on PEG-polyhydroxy copolymers, Tetronic polymers, n-isopropylacrylamide based systems may also be used. As an alternative, the drug (e.g., AVASTIN) may be dissolved in a small volume of saline solution and mixed with a concentrated solution of polymer, e.g., a 50% w/w solution of PLURONIC F-127. The ratio of mixing can be adjusted so that the drug is precipitated when the saline and polymer solution are mixed, e.g., a 1:3 ratio of drug-to-solution so that an approximately 37% solution is created. The mixture will form a gel inside the body when it is warmed to physiological temperature, so that the drug is entrapped in situ. In some embodiments, the mixture is made at a target site on or in the body, i.e., the gel is formed in situ.

In another exemplary embodiment, a biodegradable composition similar to described in the U.S. Pat. No. 6,566,406 is used. A biodegradable crosslinked matrix is formed by condensation polymerization reaction between PEG-NHS ester and trilysine. An exemplary protein based bioactive substance, fibrinogen, is mixed with the trilysine solution. The trilysine and PEG-NHS ester undergo polymerization and crosslinking leading to the formation of crosslinked hydrogel matrix. The fibrinogen mixed with dilysine undergoes is precipitated when mixed with solution containing polyethylene glycol polymer. Many proteins, especially high molecular weight proteins undergo phase separation when mixed with water soluble polymers like dextran, polyethylene glycol, polyvinyl alcohol and the like. The precipitation of fibrinogen does not affect the crosslinking reaction between dilysine and PEG-NHS ester. The precipitated fibrinogen is entrapped in the crosslinked matrix. The precipitated fibrinogen dissolves in the water and is released in the surrounding tissue. The overall release is dependent in the crosslinking density of crosslinked matrix, the dissolution rate of fibrinogen in water at physiological pH and degradation rate of crosslinked hydrogel. U.S. Pat. No. 6,566,406 teaches methods and compositions to control the degradation rate and crosslinking density of hydrogel. The dissolution of precipitated bioactive substance (fibrinogen) provides a new way to control the release of bioactive substance. When using the compositions described in U.S. Pat. No. 6,566,406, it is understood that the bioactive substance is mixed with a precursor component which will not undergo chemical reaction with the bioactive substances. In the examples described above, the compound is mixed with dilysine derivative which does not react with the fibrinogen. If the bioactive substance has chemical groups such as amine group that may react with PEG-NHS ester group, their contribution must be considered in calculating the stoichiometry between electrophilic and nucleophilic groups. The concentration of bioactive substance in the crosslinked hydrogel may range from 0.1% to 90%, more preferably 5 to 70% and even more preferably 10 to 40%. The examples discussed above causes phase in the bioactive substance by solubility stimulus. In another example, a phase change or precipitation is caused by changing the pH of the precursor compositions. In the exemplary embodiment, acidic growth factors such as bone morphogenetic protein-2 are used. Bone morphogenetic proteins (BMPs) and transforming growth factor-βs (TGF-βs) are important regulators of bone repair and regeneration. BMPs are generally dissolved in acidic solutions and precipitated when subjected to physiological pH (pH 7.2), especially when employed in high concentration. In the preferred embodiment, a reaction between electrophilic and nucleophilic precursors (PEG-NHS ester and polyamine) is controlled such that final pH upon mixing the precursors is designed to say around 7.2. At this pH, the BMP-2 mixed with the precursors in high concentration is precipitated at pH 7.2 in the crosslinked matrix. The precipitated BMP-2 is released by the hydrogel by slow dissolution to cause local therapeutic effect such as angiogenesis or bone formation.

In another embodiment, a photopolymerizable precursor such as a PEG based macromer is used, e.g., as in U.S. Pat. No. 5,410,016. In one exemplary embodiment, a PEG based macromonomer is mixed with long UV photoinitiator IRGACURE 2959. This macromer solution is then mixed with exemplary drug fibrinogen solution and irradiated with long UV light until polymerization of PEG macromer is partially or substantially complete which results in formation of hydrogel. The contact of PEG solution with fibrinogen solution forces the fibrinogen to precipitate. The dissolution of fibrinogen in the hydrogel matrix is exploited to control its release. Other bioactive substances such as BMPs, acidic and basic growth factors may also be used in similar fashion and may be forced to precipitate by contact with macromonomer or by changing the pH of resulting final solution prior to polymerization. The methods and compositions described in U.S. Pat. No. 5,410,016 can be used to control the properties of macromonomer and the crosslinked matrix.

In another embodiment, a composition is used wherein the phase change or precipitation is caused by use of organic solvents. In the exemplary embodiment, a highly water insoluble drug such as paclitaxel is dissolved in semi-aqueous solution such as ethanol solution containing macromonomer and free radical initiator. Upon polymerization of macromonomer and removal of ethanol, the paclitaxel is entrapped in the hydrogel matrix as solid. The dissolution of paclitaxel crystals leads to slow release from the crosslinked matrix. Other sparingly water soluble bioactive that can be used include statin drugs such as CERIVASTATIN, SIMVASTATIN, chlorhexidene gluconate and the like can also be used.

Other useful non-bioactive substances may be added. These include lyophilization assisting compounds, antioxidants, catalysts that accelerate in situ gelation, coloring or visualization agents, imaging agents precipitating agents like sodium chloride and the like. The amounts and type of additive added will be dependent on the bioactive substances used and gelation system chosen. Those skilled in the pharmaceutical chemistry art will be able to choose appropriate ingredients for a given formulation.

All patents, patent applications, articles, publications and references set forth herein are hereby incorporated by reference to the extent they do not contradict what is explicitly disclosed.

The following non-limiting examples are intended to illustrate some of the various hydrogel compositions, methods of preparation for such compositions, and methods to incorporate bioactive substances. These examples point to general principles for making and using the inventions.

### Materials and Equipment

Polyethylene glycol can be purchased form various sources such as Shearwater Polymers, Union Carbide, Fluka and Polysciences. Multifunctional hydroxyl and amine terminated polyethylene glycol are purchased from Shearwater Polymers, Dow Chemicals and Texaco. PLURONIC and TETRONIC series polyols can be purchased from BASF Corporation. All other reagents, solvents are of reagent grade and can be purchased from commercial sources such as Polysciences, Fluka, Aldrich and Sigma. Most of the reagents/solvents are purified/dried using standard laboratory procedures such as described Perrin et al. Small laboratory equipment and medical supplies can be purchased from Fisher or Cole-Parmer. AVASTIN (bevacizuma) is purchased from Genentech Inc.

### General Analysis

Chemical analysis for the polymers synthesized include structural determination using nuclear magnetic resonance (proton and carbon-13), infrared spectroscopy, high pressure liquid chromatography (HPLC) and gel permeation chromatography (for molecular weight determination). Thermal characterization such as melting point and glass transition temperature can be done by differential scanning calorimetric analysis. The aqueous solution properties such as micelle formation, gel formation can be determined by fluorescence spectroscopy, UV-visible spectroscopy and laser light scattering instruments.

In vitro degradation of the polymers is followed gravimetrically at 37° C., in aqueous buffered medium such as phosphate buffered saline (pH 7.2). In vivo biocompatibility and degradation life times are assessed by injecting or forming a gelling formulation directly into the peritoneal cavity of a rat or rabbit and observing its degradation over a period of 2 days to 12 months. Alternatively, the degradation is assessed by the prefabricated sterile implant made by process like by casting the crosslinker-biological fluid composition in molds. The term fluid generally refers to solutions, emulsions, suspensions, and gels. The implant is then surgically implanted within the animal body. The degradation of the implant over time is monitored gravimetrically or by chemical analysis. The biocompatibility of the implant can be assessed by standard histological techniques.

### EXAMPLE 1

Preparation of Crosslinked Biodegradable Hydrogel with Precipitated Model Protein Drug (AVASTIN): Hydrogel Prepared by Condensation Polymerization

Drug Exists as Solid Particle During in situ Gelation

1 g of 4 arm-n-hydroxysuccinimide ester of polyethylene glycol carboxymethylene-butyric acid, average molecular weight 10000 Daltons (Shearwater 4 arm CM-HBA-NS-10K) is dissolved in 4 ml PBS. Solution B is prepared by dissolving 1 gram of amine terminated polyethylene glycol (eight arm, molecular weight 20000 g/mole) 2 ml PBS. 100 mg AVASTIN is added to PEG amine solution (solution B). The AVASTIN remains insoluble in the solution B due to high concentration of PEG amine and forms a suspension/emulsion. 0.1 ml of solution A and solution B are mixed together to from a crosslinked biodegradable hydrogel. The AVASTIN remains suspended and entrapped in the hydrogel matrix. The hydrogel is suspended in 5 ml PBS and elution of AVASTIN in PBS is monitored at 37° C. over a period of 6 weeks. A fresh PBS is exchanged at 10 min, 30 min, 60 min, 2 h, 4 h, 8 h, 16 h, 24 h, 2 day, 4 days, 7 days, 14 days and 21 days. The AVASTIN eluted solution is kept at −20° until analysis. The concentration of AVASTIN in PBS is measured by standard protein assay measurements or by UV spectrophotometry.

Addition of sucrose in the range of about 5 to about 45 percent may be used to control the solubility of AVASTIN in the amine solution.

### EXAMPLE 2

Preparation of Crosslinked Biodegradable Hydrogel with Precipitated Model Protein Drug (Fibrinogen): Hydrogel Prepared by Condensation Polymerization

Precipitation of Fibrinogen by PEG Solution

0.68 grams 4 arm-n-hydroxysuccinimide ester of polyethylene glycol and carboxymethylene-butyric acid, average molecular weight 10000 Daltons (Shearwater 4 arm CM-HBA-NS-10K) is dissolved in 2.82 g 0.01M phosphate buffer at pH 4.0 and is sterile filtered. Solution B is prepared by dissolving 0.025 grams trilysine and 10 mg bovine fibrinogen (purchased from Sigma Chemicals) in 3.47 g 0.1M borate buffer at pH 9.5 with 0.1 mg/mL methylene blue for visualization. 1 ml of solution A and B are mixed to promote condensation polymerization. Trilysine and PEG-NHS ester react to form a crosslinked hydrogel. Fibrinogen in trilysine solution is seen precipitated when PEG-NHS ester solution and lysine solutions are mixed. Fibrinogen protein is not compatible with synthetic polymers such as polyethylene glycol. The precipitated fibrinogen is released from the hydrogel over a period of time. The kinetics of release is controlled by the hydrolysis crosslinked hydrogel as well as dissolution rate of precipitated Fibrinogen in water under physiological conditions. If desired the A and B can be mixed just prior to use and then can be sprayed using commercially available spraying systems such as used by DuraSeal or SprayGel systems commercialized by Confluent Surgical Inc.

In another variation of this embodiment, sucrose is added (20% final concentration) in lysine solution (solution B) to prevent dissolution of fibrinogen. In another variation, sucrose is replaced by sodium chloride at the 20% concentration.

### EXAMPLE 3

Preparation of Crosslinked Biodegradable Hydrogel with Precipitated Protein Drug (Fibrinogen): Hydrogel Prepared by Free Radical Polymerization

Part 1: Synthesis of Polyethylene Glycol Lactate Copolymer (10KL5)

30.0 g of PEG 10000, 4.3 g of dl-lactide and 30 mg of stannous octoate are charged into 100 ml Pyrex pressure sealing tube. The tube is then connected to argon gas line and sealed under argon. The tube is then immersed in oil bath maintained at 140° C. The reaction is carried out for 16 h at 140° C. The polymer from the tube is recovered by breaking the Pyrex tube. The polymer is then dissolved in 70 ml toluene and precipitated in 2000 ml cold hexane. The precipitated polymer is recovered by filtration and dried under vacuum for 1 day at 60° C. It then immediately used in the next reaction.

Part 2: End-Capping of 10KL5 with Polymerizable or Crosslinkable Group (10KL5A2)

30 g of 10KL5 is dissolved in 450 ml dry toluene. About 50 ml of toluene is distilled out to remove traces of water from the reaction mixture. The warm solution is cooled to 65° C. To this warm solution, 1.6 g of triethylamine and 1.5 g acryloyl chloride are added. The reaction mixture is then stirred for 30 minutes at 50-60° C. and filtered. The reactive precursor is precipitated by adding the filtrate to 2000 ml cold hexane. The precipitated polymer is recovered by filtration. It is then dried under vacuum for 12 h at 50° C.

Part 3: Preparation of Hydrogel Matrix with Precipitated Fibrinogen as Model Drug

Solution A is prepared by mixing 20 g 10KL5A2, 20 g sodium chloride, 80 g saline solution buffered with 1000 mM triethanol amine buffer (pH 7.4) and 100 mg IRGACURE 2959 (Ciba Specialty Chemicals) photoinitiator. Solution B is prepared by dissolving 15 g bovine fibrinogen or human fibrinogen in 80 g saline solution buffered with 1000 mM triethanol amine buffer (pH 7.4). 1 ml of solution A and B are mixed and irradiated with long wave UV lamp emitting at 360 nm (intensity 10 mW/cm2) for 2 minutes. The 10KL5A2 undergoes polymerization and crosslinking initiated by Irgacure and UV light. The mixing of A and B also causes precipitation of fibrinogen due to contact with PEG solution. The precipitated fibrinogen remains entrapped in the polymerized 10KL5A2 matrix and released slowly upon it dissolution into the surrounding aqueous medium. The release kinetic is controlled by dissolution rate of fibrinogen and degradation rate of 10KL5A2 crosslinked hydrogel.

### EXAMPLE 4

Preparation of Crosslinked Biodegradable Hydrogel with Precipitated Protein Drug (Bone Growth Factor)

Precipitation of Protein Drug by Changing pH

1.41 grams 4 arm-n-hydroxysuccinimide ester of polyethylene glycol carboxymethylene-butyric acid, average molecular weight 10000 Daltons (Shearwater 4 arm CM-HBA-NS-10K) and 100 mg of Bone Morphogenetic Protein 2 (BMP-2 protein, recombinant, Sigma catalog B3555) are mixed in a solid state as lyophilized powders and are dissolved/suspended in 2.96 ml 0.1 M HCl just prior to use. Solution B is prepared by dissolving 0.06 grams dilysine (purchased from Sigma Chemicals) in 7.2 g 0.1M borate buffer at pH 9.5 with 0.1 mg/mL methylene blue for visualization. 1 ml of solution A and B are mixed to promote condensation polymerization between dilysine and PEG-NHS ester. Dilysine and PEG-NHS ester react to form a crosslinked hydrogel. HCl is neutralized by borate buffer to form a neutral/basic solution medium. At this pH and concentration (pH>7) BMP is insoluble and precipitates in the solution prior to crosslinking. The precipitated protein is entrapped in the hydrogel. The precipitated BMP-2 is released from the hydrogel over a period of time. The kinetics of release is controlled by the hydrolysis crosslinked hydrogel as well as dissolution rate of precipitated BMP-2. Alternatively, BMP may also be mixed first dilysine solution in 0.1M HCl and reacted with CM-HBA-NS-10K with final pH upon mixing is maintained around 7.2.

### EXAMPLE 5

Preparation of Crosslinked Biodegradable Hydrogel with Precipitated Hydrophobic Drug

Precipitation of Paclitaxel by Mixing Water Miscible Organic Solvent Solution with Aqueous Solution

0.70 grams 4 arm-n-hydroxysuccinimide ester of polyethylene glycol carboxymethylene-butyric acid, average molecular weight 10000 Daltons (Shearwater 4 arm CM-HBA-NS-10K) is dissolved in 2.96 g 0.01M phosphate buffer at pH 4.0 and is sterile filtered. Solution B is prepared by dissolving 0.30 grams tetralysine and 50 mg paclitaxel in 3.64 g 25% ethanol solution in water. 1 ml of solution A and B are mixed to promote condensation polymerization. Dilysine and PEG-NHS ester react to form a crosslinked hydrogel. The reduction in ethanol content precipitates paclitaxel crystals in the crosslinked hydrogels which remain entrapped in the hydrogel. The precipitated paclitaxel is released from the hydrogel over a period of time. The kinetics of release in aqueous environment is controlled by the dissolution of paclitaxel in the water.

### EXAMPLE 6

Preparation of Crosslinked Hydrogel Containing Precipitated Paclitaxel

A polymerizable PEG based macromonomer is prepared by mixing 20 g 10KL5A2(Example 2), 20 g ethanol, 60 g saline solution buffered with 1000 mM triethanol amine buffer (pH 7.4), 100 mg Irgacure 2959 and 100 mg of paclitaxel. 1 ml of the solution/suspension is applied on rat peritoneal cavity tissue surface and polymerized in situ using UV light (360 nm). Upon polymerization, the crosslinked gel is washed with saline solution to remove initiator fragments and alcohol. The removal of alcohol causes the precipitation of paclitaxel in the aqueous environment. The paclitaxel the released from the hydrogel by dissolution of paclitaxel crystals.

### EXAMPLE 7

Preparation of Crosslinked Biodegradable Hydrogel with Precipitated Model Small Compound Drug (Paclitaxel): Hydrogel Prepared by Condensation Polymerization

Use of Hydrophobic Drugs Entrapped in a Hydrogel.

1.37 grams 4 arm-n-hydroxysuccinimide ester of polyethylene glycol carboxymethylene-butyric acid, average molecular weight 10000 Daltons (Shearwater 4 arm CM-HBA-NS-10K) is dissolved in 5.64 g 0.01M phosphate buffer at pH 4.0 and is sterile filtered. Solution B is prepared by dissolving 0.050 grams trilysine and 100 mg paclitaxel in 6.94 g 0.1M borate buffer at pH 9.5 with 0.5 mg/mL methylene blue for visualization. The paclitaxel is insoluble in borate buffer and remains suspended in the solution B. 1 ml of solution A and B are mixed to promote condensation polymerization. Trilysine and PEG-NHS ester react to form a crosslinked hydrogel. Paclitaxel in trilysine solution remains entrapped in the crosslinked hydrogel as fine particles. The paclitaxel crystals dissolve slowly in the crosslinked hydrogel matrix and are released to the surrounding tissue. The kinetics of paclitaxel release is governed by dissolution of the paclitaxel crystals. The elution of paclitaxel is monitored using a similar method described in Example 1 in PBS. The analysis of paclitaxel in the eluted samples is performed using HPLC.

### EXAMPLE 8

Preparation of Crosslinked Biodegradable Hydrogel with Suspended Model Small Compound Drug (Vitamin El): Hydrogel Prepared by Condensation Polymerization

Use of Hydrophobic Liquid Drug Entrapped in a Hydrogel

1.4 grams 4 arm-n-hydroxysuccinimide ester of polyethylene glycol carboxymethylene-butyric acid, average molecular weight 10000 Daltons (Shearwater 4 arm CM-HBA-NS-10K) is dissolved in 5.92 g 0.01M phosphate buffer at pH 4.0 and is sterile filtered. Solution B is prepared by dissolving 0.06 grams dilysine and suspending 100 mg vitamin E as a model liquid hydrophobic drug in 7.28 g 0.1M borate buffer at pH 9.5 with 0.5 mg/mL methylene blue for visualization. The vitamin E is insoluble in borate buffer and remains suspended in the solution B. 1 ml of solution A and B are mixed to promote condensation polymerization. Dilysine and PEG-NHS ester react to form a crosslinked hydrogel. Vitamin E remains entrapped in the crosslinked hydrogel as oil droplets. The Vitamin E slowly eluted from the hydrogel to the surrounding tissue.

### EXAMPLE 9

Use of Thermosensitive Gelation System to Deliver a Suspended Protein Drug.

A solution of PLURONIC F-127 is made by dissolving 4 grams of Pluronic F127 in 6 grams of PBS. The mixture is cooled to less than 10 degree C. to dissolve the polymer for 24-48 hours. After complete dissolution, 100 mg of AVASTIN lyophilized powder is added to the cold solution. The drug remains as suspended particles due to high concentration of PEG based polymer employed in the solution. The cold solution is applied on the human or animal tissue where body temperature transforms the Pluronic solution into soft hydrogel. The precipitated drug is released from the hydrogel due to dissolution of drug crystals. Other thermosensitive sensitive systems based on PEG-polyhydroxy copolymers, Tetronic polymers, n-isopropylacrylamide based polymers systems may also be used.

### EXAMPLE 10

Use of Changes in pH to Change Solubility of Agent for Delivery.

A first precursor such as a PEG modified with electrophilic groups (e.g., 4 arm-n-hydroxysuccinimide ester) is provided that is reactable with a second precursor with nucleophilic groups, e.g., a multiarmed PEG with primary amines or trilysine. A dye may optionally be included for visualization provided that can be chosen not to interfere with gelation and creation of required physical properties. The precursors are solubilized in a low pH first medium, with the pH effectively preventing reaction between the precursor functional groups, e.g., a pH 4 weak buffer. A second medium may contain a high pH buffer with a strength that creates a higher pH when mixed with the first medium. The precursors and buffers may be adjusted to complete gelation of the precursors in a desired time. Reaction would occur in situ upon mixing of the two components and application to a target tissue before the gelation is completed.

For instance, a PEG-succinimidyl glutarate and FD&C Blue #1 milled powder were dissolved in a pH 4 weak acidic buffer system containing trilysine acetate and sodium phosphate monobasic. The pH 4 buffer was mixed with a basic sodium borate/sodium phosphate buffer at pH 8.8 to achieve a final pH of about 7.6 when the buffers were mixed, with an about 30 second gelation time. The same system was used with the basic buffers at pH of about 9.4 for a less than about 5 seconds gelation time. Accordingly, in this system for example, the basic buffer solution may contain an agent soluble at high pH only (e.g., more than about 8.8). Upon mixing of the two buffers, the pH of the system reduces, and the agent precipitates out as particle with lowered solubility at physiological pH. An example of such an agent is the drug rifampicin. Rifampicin is an antibiotic drug. The solubility at pH 4 is 1.6 g/L, at pH 7 it is 0.16 g/L, at pH 10 it is 1000 g/L.

Alternatively, an agent that is soluble at low pH only (e.g., about pH 4) may be dissolved in the acidic buffer. Upon mixing of the buffers, the pH of the system would increase, and the agent precipitates out as particle with lowered solubility at physiological pH. Examples of such agents are the drugs are Timolol and Moxifloxacin. Timolol is a drug used to treat glaucoma. The solubility at pH 4 is 1000 g/L, at pH 7 it is 160 g/L, at pH 10 it is 2.4 g/L. Moxifloxacin is another antibiotic drug. The solubility at pH 4 is 11 g/L, at pH 7 it is 0.13 g/L, at pH 10 it is 0.14 g/L.

Another alternative is the preparation of a suspension of an agent and placing it in an environment, e.g., a chosen physiological environment, wherein the pH changes to a condition where the agent is more soluble to thereby direct its dissolution and release. The agent may be suspended by preparing it as a particle, e.g., by spraying, milling, or controlled precipitation. The particles are dispersed in a solution at a first pH. As the pH changes, its rate of dissolution is changed. In some embodiments the agent is suspended or stored in a weak buffer at a pH wherein it is weakly or effectively insoluble (e.g., an acidic buffer or basic buffer, depending upon the agent's properties) so that the particle does not dissolve or effectively does not dissolve during the time it is in the buffer. For agents that are relatively more soluble upon exposure to physiological medium, the particles will begin to dissolve as the pH changes after placement in a physiological environment. For instance, Timolol can be dispersed as a suspension in a pH 10 buffer and at such time as its environment is changed to a physiological pH, it will gradually dissolve. This case exemplifies providing an agent at a first pH wherein it is insoluble and allowing its environment to change to a more soluble pH.

### Various Embodiments

Accordingly, some embodiments of the invention relate to a method of delivering a bioactive substance to a patient comprising precipitating the bioactive substance as a hydrogel is formed in situ so that the hydrogel entraps the precipitated bioactive substance. Similarly, an embodiment is the hydrogel: An in-situ formed hydrogel comprising a free surface and a surface conformal to a tissue in a patient, with the hydrogel comprising a precipitated bioactive or substance. Thus a method of delivering a bioactive substance to a patient can be one comprising precipitating the bioactive substance with a hydrogel precursor and activating the precursor to form a hydrogel in situ so that the hydrogel entraps the precipitated bioactive substance. The bioactive substance may be precipitated from a first aqueous solution by mixing the first aqueous solution with a second aqueous solution that comprises a first precursor that is crosslinked to form the hydrogel. Such precipitation may be caused by exposure to the precursor. For instance, the precursor may precipitate a bioactive substance by a volume exclusion mechanism. Or the precipitation may be caused by a change in salt concentration, solvent properties, ion content, pH, or a combination of such factors chosen in light of the substance's properties and conditions for precipitating it. As explained, in some embodiments a first precursor is reacted with a second precursor to form the hydrogel. Thus the first precursor may be photopolymerized to form the hydrogel. Or the hydrogel may be formed from two precursors that are mixed to initiate crosslinking of the precursors with each other. And, for instance, the precursor or at least one of the precursors may comprise polyalkylene oxide, polyether, polyethylene glycol, dextran, or polyvinyl pyrrolidinone, or a copolymer thereof, e.g., a copolymer having at least about 40% by molecular weight of polyalkylene oxide, polyether, polyethylene glycol, dextran, or polyvinyl pyrrolidinone.

In some embodiments, an agent is dissolved in a first solution or buffer solution at a first pH and mixed with a second buffer that changes the pH such that the agent is less soluble so that the agent precipitates. Upon exposure to a physiological solution at a predetermined location in or on a patient, the agent's environment is changed to present a pH wherein the agent is more soluble to release the agent. Thus some or all of the agent in the first solution is precipitated, e.g., at least about 25%, at least about 90%, at least about 99% w/w of the agent; artisans will immediately appreciate that all the ranges and values within the explicitly stated ranges are contemplated, e.g., from about 25% to about 98%, or more than about 80%.

It is claimed:

1. A method for forming a crosslinked polymer hydrogel comprising:
mixing a solution that comprises a medicinal agent dissolved in a water miscible solvent with a water soluble precursor to form a mixture and simultaneously precipitate the medicinal agent;
depositing the mixture at a location exposed to a physiological fluid; and
polymerizing the precursor to form the crosslinked hydrogel, entrap the precipitated agent, and subsequently gradually release the agent into the physiological fluid.

2. The method of claim 1 wherein the solution is aqueous and the precursor causes the precipitation of the medicinal agent.

3. The method of claim 2 wherein the precipitation is performed by a volume exclusion mechanism.

4. The method of claim 3 wherein the precursor comprises polyethylene oxide.

5. The method of claim 1 wherein the solution is aqueous and the precipitation is caused by a changing a member of the group consisting of salt concentration, ion content, pH, and a combination thereof.

6. The method of claim 5 wherein the solution is aqueous and a salt is mixed into the mixture at a concentration that causes the precipitation of the medicinal agent by a salting-out effect.

7. The method of claim 5 wherein the solution is aqueous and a buffer is mixed into the mixture at a concentration and pH that causes the precipitation of the medicinal agent by a change in pH.

8. The method of claim 1 wherein the water miscible solvent comprises alcohol or an organic solvent and the precipitation is performed by dilution of the alcohol or organic solvent with a second solvent that is aqueous.

9. The method of claim 1 wherein the water miscible solvent comprises alcohol or organic solvent and the precipitation is performed by removal of the alcohol or organic solvent.

10. The method of claim 1 wherein the precursor is a first precursor that comprises nucleophilic functional groups and is mixed with a second precursor that comprises electrophilic groups that undergo covalent bonding with the nucleophilic functional groups to achieve the polymerization.

11. The method of claim 1 wherein the precursor is a first precursor that comprises unsaturated functional groups and is mixed with a polymerization initiator that initiates the polymerization.

12. The method of claim 1 wherein the hydrogel is formed from two precursors that react with each other to form covalent bonds and the polymerization is initiated by mixing the two precursors.

13. The method of claim 1 wherein the precursor comprises polyalkylene oxide, polyether, polyethylene glycol, dextran, polyvinyl pyrrolidinone, or a copolymer thereof having at least about 40% by molecular weight of the polyalkylene oxide, the polyether, the polyethylene glycol, the dextran, or the polyvinyl pyrrolidinone.

14. The method of claim 1 wherein the medicinal agent is water soluble.

15. A medical device comprising a crosslinked material adhesive to and conformal to a tissue of a patient that comprises a precipitated medicinal agent soluble in a physiological solution available at the tissue.

16. The medical device of claim 15 wherein the precursor comprises polyethylene oxide.

17. The medical device of claim 15 wherein the material is a hydrogel.

18. The medical device of claim 17 wherein the hydrogel comprises a salt, buffer, alcohol, or organic solvent at a concentration effective to precipitate the bioactive substance.

19. The medical device of claim 17 wherein the hydrogel comprises a polymerization reaction product of a first precursor that comprises nucleophilic functional groups and a second precursor that comprises electrophilic groups that undergo covalent bonding with the nucleophilic functional groups to achieve the polymerization.

20. The medical device of claim 17 wherein the first precursor comprises polyalkylene oxide, polyether, polyethylene glycol, dextran, polyvinyl pyrrolidinone, or a copolymer thereof having at least about 40% by molecular weight of the polyalkylene oxide, the polyether, the polyethylene glycol, the dextran, or the polyvinyl pyrrolidinone.

21. The method of claim 15 wherein the medicinal agent is water soluble.
